(19) United States
(12) Patent Application Publication
(10) Pub. No.: US 2026/0297226 A1
(43) Pub. Date: Oct. 1, 2026

(54) STYRENE PRODUCTION FROM USED TIRES AND USE WITHIN TIRE RUBBER

(71) Applicant: Bridgestone Americas Tire Operations, LLC, Nashville, TN (US)

(72) Inventors: Terrence E. HOGAN, Uniontown, OH (US); Mark W. SMALE, Hudson, OH (US); William S. NIAURA, Hudson, OH (US)

(21) Appl. No.: 19/490,982

(22) PCT Filed: Jun. 7, 2024
(86) PCT No.: PCT/US2024/033014

§ 371 Date: Dec. 8, 2025

Related U.S. Application Data
(60) Provisional application No. 63/506,752, filed on Jun. 7, 2023.

(51) Int. Cl. C08F 36/06; C07C 2/02; C07C 5/00; C07C 29/151; C08K 3/04; C10J 3/00

(52) U.S. Cl. CPC C08F 36/06; C07C 2/02; C07C 5/00; C07C 29/1518; C08K 3/04; C10J 3/00; C10J 2300/0916; C10J 2300/0946

(57) ABSTRACT

A process comprising (a) providing a feedstock that includes carbonaceous materials; (b) thermally converting the feedstock to produce a gaseous stream including carbon monoxide, hydrogen, and carbon dioxide; (c) converting at least a portion of the carbon monoxide, hydrogen, and carbon dioxide to ethanol; (d) converting at least a portion of the ethanol to ethylene; (e) reacting at least a portion of the ethylene with benzene to thereby produce ethylbenzene; and (f) dehydrogenating the ethylbenzene to form styrene.

### FIELD OF THE INVENTION

Embodiments of the present invention are directed toward a process for converting used tires to styrene. The styrene can then be copolymerized with conjugated diene monomer to produce elastomers that can be used to produce tires. In particular embodiments, the styrene is copolymerized with butadiene that is also produced from used tires.

### BACKGROUND OF THE INVENTION

Used tires include a significant amount of carbonaceous material. Recycling the carbon into other carbon-based materials presents numerous challenges due, at least in part, to the fact that a majority of the carbon is tied up in a vulcanized network. As a result, technologically efficient modes of recycling used tire is fairly limited and includes, for example, mechanically grinding the vulcanized rubber product and using the ground rubber for various uses such as filler within composites. With an eye toward carbon efficiency, other modes of recycling carbon are desired.

### SUMMARY OF THE INVENTION

One or more embodiments of the present invention provide a process comprising (a) providing a feedstock that includes carbonaceous materials; (b) thermally converting the feedstock to produce a gaseous stream including carbon monoxide, hydrogen, and carbon dioxide; (c) converting at least a portion of the carbon monoxide, hydrogen, and carbon dioxide to ethanol; (d) converting at least a portion of the ethanol to ethylene; (e) reacting at least a portion of the ethylene with benzene to thereby produce ethylbenzene; and (f) dehydrogenating the ethylbenzene to form styrene.

Other embodiments of the present invention provide a method for producing an elastomeric polymer, the method comprising (a) providing styrene monomer synthesized from constituents of a gaseous stream that is obtained from the gasification of a feedstock including carbonaceous materials; and (b) polymerizing the styrene with diene monomer to form an elastomeric polymer.

Yet other embodiments of the present invention provide a tire component comprising a cured rubber matrix with filler dispersed therein, where the cured rubber matrix includes greater than 40 wt %, or 50 wt %, or 60 wt %, or 70 wt %, or 80 wt %, or 90 wt %, or 99 wt % of a crosslinked circular synthetic rubber that is obtained by polymerizing monomer synthesized from constituents of a gaseous stream that is obtained from the gasification of a feedstock including carbonaceous materials.

Still other embodiments of the present invention provide a tire including a tire component as provided above, where the tire includes greater than 40 wt %, or 50 wt %, or 60 wt %, or 70 wt %, or 80 wt %, or 90 wt %, or 99 wt % sustainable material.

### DETAILED DESCRIPTION OF ILLUSTRATIVE EMBODIMENTS

Embodiments of the invention are based, at least in part, on the discovery of a process for consuming used tires in the production of styrene. The styrene can advantageously be copolymerized with conjugated diene monomer to produce elastomeric polymers that can be used to manufacture tire components. Embodiments of the invention therefore provide a methodology for converting used tires back to useful tires or tire components. In certain embodiments, the styrene produced by the present invention is copolymerized with conjugated diene monomer that is also produced from used tires. According to one or more embodiments, the styrene is produced by thermally decomposing used tires to form a gaseous stream. One or more components of this gaseous stream are then converted to styrene. In one or more embodiments, one or more components of the gaseous stream are converted to ethanol, and the ethanol is then converted to ethylene. The ethylene is then reacted with benzene to ultimately form styrene.

### Process Overview—Styrene Synthesis

The process of the present invention provides a feedstock, which may include used tire feedstock, and the feedstock is converted, via thermal decomposition, to a gaseous stream that includes hydrogen, carbon monoxide, and optionally carbon dioxide. This gaseous stream, which may be referred to as synthesis gas or syngas, is then converted to ethanol by using, for example, biological fermentation processes. The ethanol is then converted to ethylene by using known processes. The ethylene is then reacted with benzene to produce ethylene benzene. The ethyl benzene is then dehydrogenated to form styrene. Advantageously, the dehydrogenation step produces by-product hydrogen that can be routed back to the fermentation process and thereby improve the overall efficiencies of the process.

### Characteristics of Feedstock

In one or more embodiments, the feedstock that is thermally decomposed to form the gaseous stream may include tire feedstock from used tires, which may also be referred to as used tire feedstock or simply tire feedstock. As the skilled person appreciates, tire feedstock may include vulcanized polymer, carbon black filler, silica, resins, oils, fibrous yarn, and metal. The vulcanized polymer may include the sulfur-crosslinked residue of natural rubber and/or one or more synthetic elastomers including diene polymers and copolymers. In one or more embodiments, the used tire feedstock may include shredded or otherwise ground tires with one or more constituents of the used tire removed. For example, the tire feedstock may be treated to remove metal by methods known in the art (e.g. magnetic separation). Alternatively, or in combination therewith, the used tire feedstock may be optionally treated to remove fibrous reinforcement such as fiber yarn or cord, which the skilled person understands is often found in conjunction with the vulcanized rubber within many tire components. Alternatively, or in combination with the foregoing, the used tire feedstock may be optionally treated to remove inorganic materials such as silica filler, which the skilled person appreciates is often found in used tire components. In any event, the tire feedstock can be processed into tire shreds, tire chips, or ground or crumb rubber and fed to the thermal decomposition unit.

In one or more embodiments, the tire feedstock is characterized by relatively low amounts of metal, which low amounts may result from pre-treatment of the tire feedstock to remove at least a portion of the metal that is typically present in used tires. In one or more embodiments, following pre-treatment of the used tires to remove metal, the tire feedstock may include less than 25 wt %, in other embodiments less than 15 wt %, and in other embodiments less than 1 wt % metal based on the entire weight of the feedstock fed to thermal decomposition in accordance with the present invention.

In one or more embodiments, the tire feedstock is characterized by relatively low amounts of fibrous yarn or cord, which low amounts may result from pre-treatment of the tire feedstock to remove at least a portion of the fibrous yarn or cord that is typically present in used tires. In one or more embodiments, following pre-treatment, the tire feedstock includes less than 5 wt %, in other embodiments less than 4 wt %, in other embodiments less than 3 wt %, in other embodiments less than 2 wt %, and in other embodiments less than 1 wt % fibrous yarn or cord based on the entire weight of the feedstock fed to thermal decomposition in accordance with the present invention.

In one or more embodiments, the tire feedstock is characterized by relatively low amounts of inorganic filler (e.g. silica), which low amounts may result from pre-treatment of the tire feedstock to remove at least a portion of the inorganic filler that is typically present in used tires. In one or more embodiments, following pre-treatment, the tire feedstock includes less than 30 wt %, in other embodiments less than 20 wt %, in other embodiments less than 10 wt %, and in other embodiments less than 5 wt % inorganic filler based on the entire weight of the feedstock fed to thermal decomposition in accordance with the present invention.

In one or more embodiments, the used tire feedstock includes tire remains from passenger tires. In other embodiments, the used tire feedstock includes tire remains from non-passenger tires such as, but not limited to, truck and bus tires, off-road vehicle tires, agricultural tires, and race tires.

In one or more embodiments, the used tires are mechanically treated (e.g. ground or shredded) to form a ground or shredded material (i.e. the feedstock is ground or shredded). This ground or shredded material (i.e. the tire feedstock), which may also be referred to as crumb, may be characterized by an advantageous compacted density. For example, the feedstock may have a compacted density of greater than 640 kg/m³, in other embodiments greater than 720 kg/m³, and in other embodiments greater than 770 kg/m³, where density is determined by ASTM D 698-07.

In one or more embodiments, the feedstock provided to the thermal decomposition unit includes used tires and optionally complementary feedstock. In one or more embodiments, the complementary feedstock, which may also be referred to as co-feed, includes carbonaceous materials other than the tire feed stock. Carbonaceous material refers to any carbon material whether in solid, liquid, gas, or plasma state. Non-limiting examples of carbonaceous materials include carbonaceous liquid product, industrial liquid recycle, municipal solid waste (MSW or msw) including municipal solid waste with higher biomass content and/or with decreased recyclable material content, urban waste, agricultural material, forestry material, wood waste, construction material, vegetative material, industrial waste, fermentation waste, petrochemical coproducts, alcohol production coproducts, coal, plastics, waste plastic, coke oven tar, lignin, black liquor, polymers, waste polymers, polyethylene terephthalate (PETA), polystyrene (PS), sewage sludge, animal waste, crop residues, energy crops, forest processing residues, wood processing residues, livestock wastes, poultry wastes, food processing residues, ethanol coproducts, spent grain, spent microorganisms, municipal waste, construction waste, demolition waste, biomedical waste, hazardous waste, or their combinations. In one or more embodiments, the carbonaceous material includes biomass. In one or more embodiments, the biomass is bagasse including, but not limited to, the bagasse of sugar cane, sorghum, and guayule plant. In one or more embodiments, the feedstock includes a blend of used tires and municipal solid waste, wherein the municipal solid waste can include biomass. In other embodiments, the feedstock includes used tires and municipal solid waste that is substantially exclusive of biomass (i.e. substantially petroleum-based solid municipal waste). In yet other embodiments, the feedstock includes used tires and biomass. In still other embodiments, the feedstock includes used tires and municipal solid waste that has had most of the recyclable plastics removed (i.e. substantially exclusive of recyclable plastics). In sub-embodiments thereof, recyclable glass and metal is also substantially removed from the municipal solid waste component.

In a further embodiment, the guayule bagasse is produced as the result of a process to extract rubber and resin from the guayule plant, such as described in U.S. Publication No. 2022/0356273, which is incorporated herein by reference. Methods for the desolventization of guayule bagasse are described in U.S. Pat. No. 10,132,563, which is also incorporated herein by reference. In one or more embodiments, the guayule bagasse contains no more than 1 wt % organic solvent (based upon the total weight of the dried bagasse). In certain embodiments, the dried bagasse contains no more than 0.5 wt % organic solvent (based upon the total weight of the dried bagasse). In one or more embodiments, the dried bagasse may contain a quantity of water and higher boiling point terpenes. In certain embodiments, the total quantity of water and higher boiling point terpenes in the dried bagasse may be higher than the content of organic solvents.

In one or more embodiments, the co-feed (e.g. biomass or municipal waste) may be characterized by a compacted density of less than 600 kg/m³, in other embodiments less than 580 kg/m³, and in other embodiments less than 560 kg/m³, where density is determined by ASTM D 698-07.

The feedstock may be characterized by the amount of co-feed (e.g. biomass or municipal waste). In one or more embodiments, the feedstock includes from about 0 to about 95, in other embodiments from about 1 to about 75, and in other embodiments from about 2 to about 55 wt % co-feed with the balance including used tire. In one or more embodiments, the feedstock includes less than 95, in other embodiments less than 80, and in other embodiments less than 70 wt % co-feed. In these or other embodiments, the feedstock includes greater than 10, in other embodiments greater than 20, in other embodiments greater than 30, in other embodiments greater than 40, in other embodiments greater than 50, and in other embodiments greater than 70 wt % used tires, with the balance including complementary feedstock.

In alternative embodiments, the feedstock is substantially, and in certain embodiments exclusively, comprised of those carbonaceous materials identified above other than tire feedstock; i.e. the feedstock is substantially or exclusively comprised of the co-feed materials identified above. For example, in one or more embodiments, the feedstock includes greater than 80 wt %, in other embodiments greater than 90 wt %, and in other embodiments greater than 99 wt % municipal solid waste. Or, in other exemplary embodiments, the feedstock includes greater than 80 wt %, in other embodiments greater than 90 wt %, and in other embodiments greater than 99 wt % biomass.

### Thermal Decomposition of Feedstock

As indicated above, the feedstock (which may include tire feedstock and optionally co-feed) is thermally decomposed into gaseous streams including hydrogen, carbon monoxide, and optionally carbon dioxide by employing techniques that are generally known in the art. As the skilled person understands, these processes may include gasification processes, and it is also known that these processes can be tailored to control the chemical nature of the resulting gaseous stream. For example, the degree of combustion can be controlled by controlling the amount of oxygen present during thermal decomposition. In one or more embodiments, the step of thermal decomposition takes place in a substantially inert environment.

Processes that may be used for the thermal decomposition step may include pyrolysis or gasification reactions as disclosed in U.S. Publication Nos. 2021/0207037; 2019/0295734; 2019/0249089; 2018/0273415; 2017/0009162; 2017/0002271; 2016/0107913; 2016/0068773; 2016/0024404; 2014/0182205; 2014/0157667; and 2014/0100294, which are incorporated herein by reference.

In one or more embodiments, where the feedstock includes both tire feedstock and co-feed, the tire feedstock and the co-feed can be introduced to the same thermal decomposition unit simultaneously. For example, the tire feedstock and the co-feed can be pre-mixed at a desired ratio to form the feedstock that is fed to the thermal decomposition unit. Alternatively, separate streams of tire feedstock and co-feed can be separately and individually fed to the thermal decomposition unit at a desired rate. In yet other embodiment, the two feedstocks (i.e. the tire feedstock and the co-feed) can be sequentially treated within the same thermal decomposition unit. In still other embodiments, the two feedstocks (i.e. the tire feedstock and the co-feed) can be treated within separate thermal decomposition units operating in parallel, and then the gaseous streams produced by the respective units can be combined to attain the desired ratio of gaseous constituents.

### Characteristics of Gaseous Product Stream

As indicated above, the gaseous product stream produced by thermal decomposition of the feedstock includes carbon monoxide, hydrogen and optionally carbon dioxide. In one or more embodiments, the gaseous product stream includes from about 5 to about 50, or in other embodiments from about 7 to about 25, or in other embodiments from about 8 to about 15 volume percent carbon dioxide. In these or other embodiments, the gaseous product stream includes from about 10 to about 85, or in other embodiments from about 20 to about 65, or in other embodiments from about 25 to about 45 volume percent hydrogen. In these or other embodiments, the gaseous product stream includes from about 20 to about 85, or in other embodiments from about 30 to about 75, or in other embodiments from about 40 to about 60 volume percent carbon monoxide. In one or more embodiments, the gaseous product stream produced by thermal decomposition includes from about 40 to about 80 wt %, in other embodiments from about 45 to about 75 wt %, and in other embodiments from about 50 to about 70 wt % carbon (i.e. carbon within carbon-based compounds) based on the total weight of the gaseous product stream.

### Conditioning of Gaseous Stream

In one or more embodiments, the gaseous stream is conditioned (i.e. treated) prior to converting the gaseous stream to ethanol. In one or more embodiments, the gaseous product stream from thermal decomposition may be pressurized. In one or more embodiments, pressurization of the gaseous stream achieves sufficient pressure to overcome counter forces within the bioreactor. As the skilled person understands, this will permit flow of the gas through the bioreactor and allow inert gases (e.g. nitrogen) within the gaseous stream to enter the head space of the reactor. In one or more embodiments, the gaseous stream is pressurized to a pressure of from about 5 to about 20 barr.

Also, the gaseous stream can be cooled. As the skilled person will appreciate, the gaseous stream can be cooled, for example, within a heat exchanger such as a water-cooled unit. In one or more embodiments, the gaseous stream is cooled to a temperature below that which would otherwise have a deleterious impact on the microorganism culture within the bioreactor. In one or more embodiments, the gaseous stream is cooled to a temperature of from about 25 to about 45° C. prior to delivery to the bioreactor.

Still further, the gaseous stream can be treated to remove undesirable constituents that may be entrained within the stream. For example, the gaseous stream can be treated within a scrubber prior to being introduced to the bioreactor. In one or more embodiments, this may include the use of a catalyst (e.g. iron oxide) to remove sulfur compounds such as hydrogen sulfide. The stream may also be treated to remove acids (e.g. treatment with calcium or sodium carbonate with particular interest in removing hydrogen cyanides). Exemplary systems for removing hydrogen sulfide from gaseous streams include those available from EcoVapor (Denver, Colorado). Other methods include the use of caustic such as those systems available from DMT under the tradename Sulfurex BF (Netherlands).

### Syngas to Ethanol

As indicated above, constituents of the gaseous stream are converted to ethanol that is contained within an ethanol-containing stream; i.e. a product stream that includes ethanol. In one or more embodiments, the gaseous stream is supplemented with hydrogen prior to converting the gaseous stream to an ethanol-containing stream.

In one or more embodiments, the gaseous stream is converted to ethanol via biosynthetic techniques. For example, it is known that syngas can be converted to ethanol by fermentation utilizing microorganisms, such as bacteria. The microorganisms may be acetogenic autotrophic microbes. Acetogenic microbes (i.e. bacteria) generally transform the CO, H₂, and CO₂ in syngas into acetyl-CoA. The acetyl-CoA is then converted to organic products, such as acetic acid and ethanol. In one or more embodiments of the present invention, it may be desirable to preferentially produce ethanol relative to acetic acid.

As another potential pathway, some microorganisms (e.g. acetogens) can reduce the acetic acid (e.g. an organic acid) into an alcohol (e.g. ethanol). This acetogenic mechanism, as well as the microorganism chosen, may be considered relative to preferentially producing ethanol. Microorganisms other than acetogens may be suitable with different pathways to reaching ethanol. One or more useful microorganisms may simultaneous uptake both CO and H₂ in the syngas. In these or other embodiments, certain microorganisms can reduce CO2 to CO in the presence of excess hydrogen. For example, useful microorganisms and techniques for their use are disclosed in “A Techno-Economic Assessment of Bioethanol Production from Switchgrass Through Biomass Gasification and Syngas Fermentation” Regis et al, Energy 274, 127318, (2023).

The skilled person also appreciates that various conditions of microorganism-driven biosynthesis may be adjusted relative to preferential production of ethanol. For example, the pH, temperature, and concentrations of nutrients within the bioreactor where in the biosynthesis takes place can be adjusted. The desirable pH, temperature, and concentrations of nutrients may depend on the particular microorganisms employed.

In other embodiments, the syngas is converted to ethanol via thermochemical techniques. The skilled person understands that several thermochemical techniques exist to convert syngas to ethanol. For example, two-step processes exist whereby syngas can be converted to methanol using a hydrogen to carbon monoxide ratio of 2:1. These reactions typically take place in the gas phase using copper-based catalysts. The resulting product stream, which is typically saturated with water, can be purified by using known distillation techniques. The methanol can then be catalytically converted to ethanol. One-step catalytic techniques are also known. An exemplary thermochemical process for converting syngas to ethanol is described in U.S. Pat. No. 9,115,046, which is incorporated herein by reference.

The resultant ethanol is contained with an ethanol-containing stream. According to embodiments of the invention, a crude ethanol-containing stream, which is obtained directly from the reactor (e.g. bioreactor) in which the ethanol is synthesized, is delivered to downstream steps. In other embodiments, the ethanol-containing stream is purified to produce an ethanol-containing stream that is higher in ethanol content. In one or more embodiments, the ethanol-containing stream exiting the step wherein syngas is converted to ethanol (e.g. the bioreactor) and delivered to the downstream steps where ethanol is further converted (optionally after purification) includes greater than 80 wt %, in other embodiments greater than 90 wt %, and in other embodiments greater than 95 wt % ethanol based upon the entire weight of the ethanol-containing stream.

### Ethanol to Ethylene

As indicated above, ethanol within the ethanol-containing stream is converted to ethylene, which is then contained within an ethylene-containing stream. In one or more embodiments, ethanol is converted to ethylene using catalytic techniques, which are well known in the art. Examples of useful catalysts include acid catalysts, alumina and transition metal oxides, silicoaluminophosphates (SAPO), HZSM-5 zeolite catalyst, and heteropolyacid catalysts. Modifications of these catalysts, such as nanoscale versions, may also be employed. As the skilled person appreciates, some catalytic techniques proceed by dehydrating ethanol to form ethylene. For example, acid catalyst first protonates the hydroxyl group of the ethanol forming a molecule of water as a leaving group. The conjugate base of the remaining catalyst then deprotonates the methyl group, and the hydrocarbon rearranges to ethylene. In other embodiments, ethanol is converted to ethylene via catalytic dehydration over an aluminum oxide catalyst. In one or more embodiments, conversion of ethanol to ethylene takes place at a temperature of from about 180° C. to about 500° C.

The skilled person appreciates that processes for the conversion of ethanol to ethylene are well known and commercially available. For example, commercial processes are marketed by Braskem S.A., Technip Energies, Axens S.A. and Scientific Design Company, Inc.

The resultant ethylene is contained with an ethylene-containing stream. According to embodiments of the invention, a crude ethylene-containing stream, which is obtained directly from the reactor in which the ethylene is synthesized, is delivered to downstream steps. In other embodiments, the ethylene-containing stream is purified to produce an ethylene-containing stream that is higher in ethylene content. In one or more embodiments, the ethylene-containing stream exiting the step wherein ethanol is converted to ethylene and delivered to the downstream steps where ethylene is further converted (optionally after purification) includes greater than 80 wt %, in other embodiments greater than 90 wt %, and in other embodiments greater than 95 wt % ethylene based upon the entire weight of the ethylene-containing stream.

### Ethylene Reacted with Benzene

As indicated above, ethylene within the ethylene-containing stream is reacted with benzene to produce ethylbenzene. The skilled person understands that benzene can be alkylated with ethylene by employing known techniques. For example, streams of benzene and ethylene can be combined and reacted in the presence of an appropriate catalyst. Useful catalysts include aluminosilicate zeolites such as the Zeolite Socony Mobil-5 catalyst (i.e. ZSM-5). These reactions can take place in the gas phase using fixed-bed catalysts. In order to maintain a desirable polymerization medium, these reactions are typically maintained under pressure (e.g. pressures as high as 10 atmospheres), and may be conducted at temperatures of from about 200 to about 400° C. The synthesis of ethylbenzene by reacting ethylene with benzene is generally known as described in U.S. Pat. Nos. 4,547,605; 4,016,218; 4,891,458; and 5,334,795.

In one or more embodiments, a crude ethylbenzene-containing stream obtained directly from the reactor is delivered downstream for further conversion. In other embodiments, the ethylbenzene-containing stream is purified to produce a ethylbenzene-containing stream that is higher in ethylbenzene content. In one or more embodiments, the ethylbenzene-containing stream exiting the step wherein ethylene is reacted with benzene and delivered to the downstream steps (optionally after purification) includes greater than 80 wt %, in other embodiments greater than 90 wt %, and in other embodiments greater than 95 wt % ethylbenzene based upon the entire weight of the ethylbenzene-containing stream.

### Ethylbenzene to Styrene

In one or more embodiments, ethylbenzene within the ethylbenzene-containing stream is dehydrogenated to produce styrene and by-product hydrogen. The skilled person understands that the dehydrogenation of ethylbenzene can be catalyzed by known techniques. For example, iron(ii) oxide can catalyze the dehydrogenation reaction at elevated temperatures (e.g. about 540 to about 675° C.) in the presence of water. It is also known to used mixed catalyst systems, such as those that include potassium oxide, to prevent coking.

In one or more embodiments, a crude styrene-containing stream obtained directly from the reactor is delivered downstream for further use (e.g. polymerization). In other embodiments, the styrene-containing stream is purified to produce a styrene-containing stream that is higher in styrene content. In one or more embodiments, the styrene-containing stream exiting the step wherein ethylbenzene is converted to styrene and delivered to the downstream steps (optionally after purification) includes greater than 80 wt %, in other embodiments greater than 90 wt %, and in other embodiments greater than 95 wt % styrene based upon the entire weight of the styrene-containing stream.

### By-Product Hydrogen Recycle

As indicated above, dehydrogenation of the ethylbenzene produces by-product hydrogen that can be routed to and used within the step of converting syngas to ethanol. For example, and as discussed above, thermochemical processes to convert syngas to ethanol include reacting hydrogen with carbon dioxide at a 2:1 ratio to form methanol, which is then converted to ethanol. This is particularly advantageous in the present invention since used tires can be the primary feedstock, and used tires include a higher molar ratio of carbon to hydrogen than do other feedstock such as biomass. The skilled person understands that hydrogen can be separated from other gases within the product stream by using various techniques including pressure swing absorption.

### INDUSTRIAL APPLICABILITY

In one or more embodiments, the styrene monomer produced by the methods of this invention can be used in the production of elastomeric polymers. For purposes of this specification, these polymers may be referred to as circular synthetic rubber. In one or more embodiments, this circular synthetic rubber can be used in the manufacture of tire components. As a result, practice of the present invention provides a method by which waste material, in particular that waste material from used tires, is converted back to useful tires. In other words, a tire recycling or tire circularity method is provided.

The elastomeric polymers that can be produced by practice of the invention, which may also be referred to as styrene-containing elastomers, include, but are not limited to, poly(styrene-co-butadiene), poly(styrene-co-isoprene), and poly(styrene-co-isoprene-co-butadiene).

The synthesis of elastomeric polymers is well known and can be accomplished by using several synthetic routes (i.e. polymerization mechanisms and techniques). For example, the blend of monomer (i.e. styrene and diene monomer) can be polymerized by free-radical emulsion polymerization, anionic polymerization, or coordination catalysis using, for example, neodymium-based catalyst systems.

In one or more embodiments, the diene monomer (e.g. butadiene monomer) that is copolymerized with the styrene (obtained my practice of this invention) is also obtained from used tires as described, for example, in co-pending PCT/US2022/081188, which is incorporated herein by reference.

In one or more embodiments, other sustainably-sourced styrene is combined with the styrene of the present invention to produce the elastomeric polymers of the present invention. For example, styrene can be obtained from bio-synthesized feedstock, such as bioethanol, that is subsequently converted to styrene; see, e.g. U.S. Pat. No. 9,663,445. Alternatively, styrene can be synthesized from bio-based materials such as cinnamic acid or hydrocinnamic acid; see, U.S. Pat. No. 9,868,853. Yet other examples include styrene obtained from the depolymerization of polystyrene from post-consumer waste; see U.S. Publication No. 2022/0411351. The skilled person also understands that styrene can be obtained from those processes that are mass balanced to qualify as bio-based, bio-circular, or circular as designated by the International Sustainability and Carbon Certification ISCC. In other embodiments, the styrene of the present invention is combined with fossil-derived styrene to produce the elastomeric polymers of the present invention.

It should be appreciated that the elastomeric polymers prepared by polymerizing the styrene of the present invention, together with diene monomer, have a relatively high percentage of styrene mer units deriving from the styrene produced by the invention, and therefore the elastomeric polymers of this invention have a relatively high sustainable content. This is especially the case where the comonomer (i.e. the diene monomer) is also sourced from sustainable sources. In one or more embodiments, greater than 50 mol %, in other embodiments greater than 60 mol %, in other embodiments greater than 70 mol %, in other embodiments greater than 80 mol %, in other embodiments greater than 90 mol %, in other embodiments greater than 95 mol %, and in other embodiments greater than 99 mol % of the styrene mer units derive from the polymerization of styrene produced by this invention (i.e. styrene monomer that is synthesized from a gaseous stream obtained by gasification of carbonaceous materials). Likewise, where the elastomeric polymers are synthesized by copolymerizing the styrene of this invention with other sustainably-derived comonomer, resulting elastomeric polymer has a relatively high sustainable content. In one or more embodiments, the elastomeric polymers of the present invention together with sustainable comonomer include greater than 50 mol %, in other embodiments greater than 60 mol %, in other embodiments greater than 70 mol %, in other embodiments greater than 80 mol %, in other embodiments greater than 90 mol %, in other embodiments greater than 95 mol %, and in other embodiments greater than 99 mol % sustainable mer units (i.e. styrene mer units that are obtained from monomer synthesized from a gaseous stream obtained by gasification of carbonaceous materials and other sustainable comonomer).

It will also be appreciated that the polymers produced by this invention can be functionalized. For example, it is known that styrene-containing elastomers, such as SBR, can be modified to include filler-interactive substituents. These substituents can be imparted to the polymer using various techniques including, but not limited to, reacting the reactive polymer with an end-functionalizing agent prior to quenching the polymerization. These end-functionalizing agents may include compounds such as, but not limited to, imines, cyclic amines, and alkoxy silanes. Exemplary functionalizing agents are disclosed in, for example, U.S. Pat. Nos. 9,296,832; 9,127,109; 9,062,017; 8,962,745; and Publication No. US 2015/0274944, which are incorporated herein by reference. The polymers can also be polymerized by using functionalized initiators as disclosed, for example, in U.S. Pat. No. 9,884,923, which is incorporated herein by reference.

The elastomeric polymers produced by the present invention exhibit excellent viscoelastic properties and are particularly useful in the manufacture of various tire components including, but not limited to, tire treads, sidewalls, subtreads, and bead fillers. These elastomeric polymers can be used as all or part of the elastomeric component of a tire stock. When the elastomeric polymers produced by the present invention are used in conjunction with other vulcanizable polymers to form the elastomeric component of a tire stock, these other vulcanizable polymers may include natural rubber, synthetic rubbers, and mixtures thereof. Examples of synthetic rubber include polyisoprene, poly(styrene-co-butadiene), and other polybutadienes, poly(styrene-co-butadiene-co-isoprene), and mixtures thereof. The polymers of this invention can also be used in the manufacture of hoses, belts, shoe soles, window seals, other seals, vibration damping rubber, and other industrial products.

Practice of the present invention not only offers a method for recycling tires by employing used tires as a feedstock to produce polymer that can be formulated back into tires, but the practice of the present invention also advantageously provides a method whereby a tire is produced that has a relatively high content of sustainable constituents, which include recycled materials, naturally-derived materials and/or materials synthesized from bio-synthesized feedstock or bio-based materials. Moreover, these tires or tire components include threshold amounts of circular synthetic rubber while being characterized by high sustainable content. For example, the tires or tire components of the present invention can include greater than 40 wt %, in other embodiments greater than 50 wt %, and in other embodiments greater than 60 wt % sustainable materials. In these or other embodiments, the tire or tire components include from about 40 to about 90 wt %, in other embodiments from about 45 to about 85 wt %, and in other embodiments from about 50 to about 80 wt % sustainable material. In combination therewith, the rubber component of the tires or tire components of the present invention include greater than 10 wt %, in other embodiments greater than 20 wt %, in other embodiments greater than 30 wt %, in other embodiments greater than 40 wt %, in other embodiments greater than 45 wt %, and in other embodiments greater than 50 wt % circular synthetic rubber, which includes synthetic rubber produced according to embodiments of the present invention.

As the skilled person understands, tire components are produced by preparing and vulcanizing rubber compositions, which are often referred to as vulcanizable rubber compositions or simply rubber compositions. Generally, the vulcanizable rubber compositions include a rubber component, filler, wax, and curative. Other ingredients that may be included in the vulcanizable rubber composition include extender oils, processing oils, resins, and antidegradants.

### Elastomer

The rubber component of the vulcanizable rubber compositions includes a vulcanizable elastomer, which may also be referred to as an elastomer, a vulcanizable rubber, or simply as a rubber. The skilled person appreciates that the vulcanizable elastomer is capable of being cured, which may also be referred to as vulcanized, to form an elastomeric composition. In one or more embodiments of this invention, the rubber component includes one or more synthetic polymers. These synthetic polymers may include, for example and without limitation, synthetic polyisoprene, polybutadiene, polyisobutylene-co-isoprene, neoprene, poly(ethylene-co-propylene), poly(styrene-co-butadiene), poly(styrene-co-isoprene), poly(styrene-co-isoprene-co-butadiene), poly(isoprene-co-butadiene), poly(ethylene-co-propylene-co-diene), polysulfide rubber, acrylic rubber, urethane rubber, silicone rubber, epichlorohydrin rubber, and mixtures thereof. These elastomers can have a myriad of macromolecular structures including linear, branched, and star-shaped structures.

In these or other embodiments, the rubber component of the vulcanizable rubber compositions of this invention includes one or more circular synthetic rubbers, which is vulcanizable rubber produced from used tires. These circular synthetic rubbers may be used alone as the rubber component or in combination with synthetic polymers.

In these or other embodiments, the rubber component may include one or more natural rubbers. Natural rubber may be used alone as the rubber component or in combination with synthetic polymers and/or circular synthetic rubber. As the skilled person understands, natural rubber is synthesized by and obtained from plant life. For example, natural rubber can be obtained from Hevea rubber trees, guayule shrub, gopher plant, mariola, rabbitbrush, milkweeds, goldenrods, pale Indian plantain, rubber vine, Russian dandelions, mountain mint, American germander, and tall bellflower.

Generally, the rubber compositions of this invention include from about 30 to about 65 wt %, in other embodiments from about 35 to about 60 wt %, and in other embodiments from about 40 to about 55 wt % elastomer, based on the total weight of the tire component.

### Filler

As suggested above, the rubber compositions include fillers such as organic and inorganic fillers. Examples of organic fillers include carbon black and starch. Examples of inorganic fillers include silica, aluminum hydroxide, magnesium hydroxide, mica, talc (hydrated magnesium silicate), and clays (hydrated aluminum silicates). In certain embodiments, a mixture of different fillers may be advantageously employed.

The amount of total filler employed in the rubber compositions can be up to about 150 parts by weight per 100 parts by weight of rubber (phr), with about 30 to about 125 phr, or about 40 to about 110 phr being typical. In certain embodiments the total filler content is greater than about 100 phr. In other embodiments, the total filler content is from about 50 to about 100 phr, and in in further embodiments from about 55 to about 95 phr.

Conventional carbon black can be used, which is generally known in the art. In one or more embodiments, carbon blacks include furnace blacks, channel blacks, and lamp blacks. More specific examples of carbon blacks include super abrasion furnace blacks, intermediate super abrasion furnace blacks, high abrasion furnace blacks, fast extrusion furnace blacks, fine furnace blacks, semi-reinforcing furnace blacks, medium processing channel blacks, hard processing channel blacks, conducting channel blacks, and acetylene blacks.

In particular embodiments, the carbon blacks may have a surface area (EMSA) of at least 20 m²/g and in other embodiments at least 35 m²/g; surface area values can be determined by ASTM D-1765 using the cetyltrimethylammonium bromide (CTAB) technique. The carbon blacks may be in a pelletized form or an unpelletized flocculent form. The preferred form of carbon black may depend upon the type of mixing equipment used to mix the rubber compound.

In one or more embodiments, carbon black can be sourced from a recycled material. Such recycled material can include reclaimed or recycled vulcanized rubber, whereby the vulcanized rubber is typically reclaimed from manufactured articles such as a pneumatic tire, an industrial conveyor belt, a power transmission belt, and a rubber hose. The recycled carbon black may be obtained by a pyrolysis process or other methods known for obtaining recycled carbon black. In an aspect, a recycled carbon black can be formed from incomplete combustion of recycled rubber feedstock or rubber articles. In another aspect, the recycled carbon black can be formed from the incomplete combustion of feedstock including oil resulting from the tire pyrolysis process. The carbon blacks utilized in the preparation of the vulcanizable elastomeric compositions can be in pelletized form or an unpelletized flocculent mass.

The amount of carbon black employed in the rubber compositions can be up to about 75 parts by weight per 100 parts by weight of rubber (phr), with about 5 to about 60 phr, or about 10 to about 55 phr being typical.

The rubber composition can further include filler in the form of one or more recycled rubbers in a particulate form. Recycled particulate rubber is typically broken down and reclaimed (or recycled) by any of a plurality of processes, which can include physical breakdown, grinding, chemical breakdown, devulcanization, cryogenic grinding, a combination thereof, etc. The term recycled particulate rubber can relate to both vulcanized and devulcanized rubber, where devulcanized recycle or recycled rubber (reclaim rubber) relates to rubber which has been vulcanized, ground into particulates and may have further undergone substantial or partial devulcanization. In an example, the recycled particulate rubber used in the rubber composition is essentially free of recycled rubber resulting from devulcanization. In a situation where the vulcanized rubber contains wire or textile fiber reinforcement, such wire or fiber reinforcement can be removed by any suitable process such as magnetic separation, air aspiration and/or air flotation step. In certain embodiments, the “recycled particulate rubber” comprises cured, i.e., vulcanized (crosslinked) rubber that has been ground or pulverized into particulate matter having a mean average particle size as discussed below.

Certain silicas may be considered sustainable materials. Some commercially available silicas which may be used as sustainable materials for the current invention include Hi-Sil™ 215, Hi-Sil™ 233, and Hi-Sil™ 190 (PPG Industries, Inc. ; Pittsburgh, Pa.). Other suppliers of commercially available silica include Grace Davison (Baltimore, Md.), Degussa Corp. (Parsippany, N.J.), Rhodia Silica Systems (Cranbury, N.J.), and J.M. Huber Corp. (Edison, N.J.). Other sustainable silicas include those derived from rice husk ash.

In one or more embodiments, silicas may be characterized by their surface areas, which give a measure of their reinforcing character. The Brunauer, Emmet and Teller (“BET”) method (described in J. Am. Chem. Soc., 1939, vol. 60, 2 p. 309-319) is a recognized method for determining the surface area. The BET surface area of silica is generally less than 450 m²/g. Useful ranges of surface area include from about 32 to about 400 m²/g, about 100 to about 250 m²/g, and about 130 to about 240 m²/g, and about 170 to about 220 m²/g. In certain embodiments, the silica may have a BET surface area of 190 to about 280 m²/g. The pH's of the silicas are generally from about 5 to about 7 or slightly over 7, or in other embodiments from about 5.5 to about 6.8.

In one or more embodiments, where silica is employed as a filler (alone or in combination with other fillers), a coupling agent and/or a shielding agent may be added to the rubber compositions during mixing in order to enhance the interaction of silica with the elastomers. Useful coupling agents and shielding agents are disclosed in U.S. Pat. Nos. 3,842,111; 3,873,489; 3,978,103; 3,997,581; 4,002,594; 5,580,919; 5,583,245; 5,663,396; 5,674,932; 5,684,171; 5,684,172; 5,696,197; 6,608,145; 6,667,362; 6,579,949; 6,590,017; 6,525,118; 6,342,552; and 6,683,135; which are incorporated herein by reference.

The amount of silica employed in the rubber compositions can be from about 1 to about 150 phr or in other embodiments from about 5 to about 130 phr. The useful upper range is limited by the high viscosity imparted by silicas. In certain embodiments, the silica employed in the rubber composition is derived from rice husk ash only, and in other embodiments the rubber compositions do not include silica from non-rice husk ash derived processes. When silica is used together with carbon black, the amount of the silica or carbon black individually can be as low as about 1 phr. Generally, the amounts of coupling agents and shielding agents range from about 4 wt % to about 20 wt % based on the weight of silica used. In one or more embodiments, where carbon black and silica are employed in combination as a filler, the weight ratio or silica to total filler may be from about 5 wt % to about 99 wt % of the total filler, in other embodiments from about 10 wt % to about 90 wt % of the total filler, or in yet other embodiments from about 50 wt % to about 85 wt % of the total filler. In certain embodiments the silica and carbon black fillers employed in the rubber composition are selected from the group consisting of sustainable pyrolysis carbon black and/or rice husk ash derived silica.

### Curative

A multitude of rubber curing agents (also called vulcanizing agents) may be employed, including sulfur or peroxide-based curing systems. Curing agents are described in Kirk-Othmer, ENCYCLOPEDIA OF CHEMICAL TECHNOLOGY, Vol. 20, pgs. 365-468, (3^{rd} Ed. 1982), particularly Vulcanization Agents and Auxiliary Materials, pgs. 390-402, and A. Y. Coran, Vulcanization, ENCYCLOPEDIA OF POLYMER SCIENCE AND ENGINEERING, (2^{nd} Ed. 1989), which are incorporated herein by reference. Vulcanizing agents may be used alone or in combination.

### Wax

In one or more embodiments, the rubber composition may include one or more natural waxes. A natural wax, or one with no petroleum as its raw material, can include carnauba wax, candelilla wax (e.g., extracted from candelilla flowers), rice wax (e.g., separated from rice bran oil) and Japan wax (e.g., extracted from Japanese wax tree). In these or other embodiments, the natural wax may be used in conjunction petroleum-based waxes.

In one or more embodiments, the rubber compositions of this invention include threshold amounts of sustainable waxes, which for purposes of this invention include circular synthetic waxes and natural waxes. The amount of sustainable wax, relative to the total weight of wax included, may be from about 1 wt % to about 99 wt %, or in other embodiment from about 20 wt % to about 80 wt % of the total wax. In certain embodiments, the rubber composition includes sustainable waxes only.

### Oils

With regard to oils, sustainable oils, which include plant-based oils and bio-based oils, may be used. Plant-based oils may include plant-based triglycerides. Exemplary oils include, without limitation, palm oil, soybean oil (also referred to herein as soy oil), rapeseed oil, sunflower seed, peanut oil, cottonseed oil, oil produced from palm kernel, coconut oil, olive oil, corn oil, grape seed oil, hemp oil, linseed oil, rice oil, safflower oil, sesame oil, mustard oil, flax oil. Other examples include nut-derived oils such oils obtained from beech nuts, cashews, mongongo nuts, macadamia nuts, pine nuts, hazelnuts, chestnuts, acorns, almonds, pecans, pistachios, walnuts, or brazil nuts. As the skilled person will appreciate, these oils can be produced by any suitable process such as mechanical extraction (e.g., using an oil mill), chemical extraction (e.g., using a solvent, such as hexane or carbon dioxide), pressure extraction, distillation, leaching, maceration, purification, refining, hydrogenation, sparging, etc.

Bio-based oils, also referred to as bio-oils, can include oils produced by a recombinant cell. For example, bio-oils produced by recombinant cells can be produced using a select strain of algal cells that are fed with a supply of sugars (e.g., sucrose) and then allowed to ferment and produce a bio-oil with a selected profile; after sufficient growth or fermentation has taken place, the bio-oil is isolated from the cells and collected.

Generally, the rubber compositions of this invention can include from about 1 to about 70 parts by weight, or in other embodiments from about 5 to about 50 parts weight total oil per 100 parts by weight rubber. The amount of sustainable oil, relative to the total weight of oil included, may be from about 1 wt % to about 99 wt %, or in other embodiment from about 20 wt % to about 80 wt %.

### Other Ingredients

Other ingredients that are typically employed in rubber compounding may also be added to the rubber compositions. These include accelerators, accelerator activators, oils, plasticizer, waxes, scorch inhibiting agents, processing aids, zinc oxide, tackifying resins, reinforcing resins, fatty acids such as stearic acid, peptizers, and antidegradants such as antioxidants and antiozonants.

### Rubber Composition Processing

All ingredients of the rubber compositions can be mixed with standard mixing equipment such as Banbury or Brabender mixers, extruders, kneaders, and two-rolled mills. In one or more embodiments, the ingredients are mixed in two or more stages. In the first stage (often referred to as the masterbatch mixing stage), a so-called masterbatch, which typically includes the rubber component and filler, is prepared. To prevent premature vulcanization (also known as scorch), the masterbatch may exclude vulcanizing agents. The masterbatch may be mixed at a starting temperature of from about 25° C. to about 125° C. with a discharge temperature of about 135° C. to about 180° C. Once the masterbatch is prepared, the vulcanizing agents may be introduced and mixed into the masterbatch in a final mixing stage, which is typically conducted at relatively low temperatures so as to reduce the chances of premature vulcanization. Optionally, additional mixing stages, sometimes called remills, can be employed between the masterbatch mixing stage and the final mixing stage. One or more remill stages are often employed where the rubber composition includes silica as the filler. Various ingredients including the polymers of this invention can be added during these remills.

The mixing procedures and conditions particularly applicable to silica-filled tire formulations are described in U.S. Pat. Nos. 5,227,425; 5,719,207; and 5,717,022, as well as European Patent No. 890,606, all of which are incorporated herein by reference. In one embodiment, the initial masterbatch is prepared by including the polymer and silica in the substantial absence of coupling agents and shielding agents.

### Tire Fabrication

In order to fabricate tire components with the polymers produced by the invention, the skilled person appreciates that the polymers are mixed with various other ingredients (e.g. filler and curative) to produce a rubber composition (also referred to as vulcanizable composition), and the vulcanizable composition is then processed into tire components according to ordinary tire manufacturing techniques, which generally include standard rubber shaping and molding techniques. The tire components may include, but are not limited to, tire treads, sidewalls, subtreads, body ply skims, and bead filler. The various tire components are then assembled into a green tire (i.e. an uncured tired), placed within a mold, and then vulcanized. Typically, vulcanization is effected by heating the vulcanizable composition in a mold; e.g., it may be heated to about 140° C. to about 180° C. Cured or crosslinked rubber compositions may be referred to as vulcanizates, which generally contain three-dimensional polymeric networks that are thermoset. The other ingredients, such as fillers and processing aids, may be evenly dispersed throughout the crosslinked network. Pneumatic tires can be made as discussed in U.S. Pat. Nos. 5,866,171; 5,876,527; 5,931,211; and 5,971,046, which are incorporated herein by reference.

### Tire Reinforcements

In one or more embodiments, the tires can include fabric reinforcement made by using non-petroleum materials in place of synthetic fibers. For example, mechanical recycled fibers, chemical recycled fibers, or bio-based fibers can be used. Likewise, the tires can include metal reinforcement made from recycled steel and/or other circular or sustainable metals. These non-petroleum fabrics and recycled metals can be used exclusively within the tires or in combination with traditional fabric and/or metal reinforcement.

Various modifications and alterations that do not depart from the scope and spirit of this invention will become apparent to those skilled in the art. This invention is not to be duly limited to the illustrative embodiments set forth herein.

1. A process comprising:
(a) providing a feedstock that includes carbonaceous materials;
(b) thermally converting the feedstock to produce a gaseous stream including carbon monoxide, hydrogen, and carbon dioxide;
(c) converting at least a portion of the carbon monoxide, hydrogen, and carbon dioxide to ethanol;
(d) converting at least a portion of the ethanol to ethylene;
(e) reacting at least a portion of the ethylene with benzene to thereby produce ethylbenzene; and
(f) dehydrogenating the ethylbenzene to form styrene.

2. The process of claim 1, where said step of dehydrogenating the ethylbenzene to form styrene produces hydrogen gas as a by-product, and further comprising the step of directing the hydrogen gas to said step of converting at least a portion of the carbon monoxide, hydrogen, and carbon dioxide to ethanol.

3. The process of claim 1, where said step of thermally converting the feedstock includes gasifying the feedstock.

4. The process of claim 1, where said the step of converting at least a portion of the carbon monoxide, hydrogen, and carbon dioxide to ethanol includes biosynthetically converting at least a portion of the carbon monoxide, hydrogen, and carbon dioxide to ethanol, or where said the step of converting at least a portion of the carbon monoxide, hydrogen, and carbon dioxide to ethanol includes thermochemically converting at least a portion of the carbon monoxide, hydrogen, and carbon dioxide to ethanol.

5. The process of claim 1, where said step of converting at least a portion of the ethanol to ethylene includes catalytically dehydrating the ethanol.

6. The process of claim 1, further comprising the step of providing a diene comonomer, and polymerizing the styrene with the diene comonomer to produce a elastomeric polymer.

7. The process of claim 6, where the diene comonomer is a sustainable diene comonomer.

8. The process of claim 1, where the feedstock includes used tire feedstock, biomass, municipal solid waste, or a mixture of tire feedstock and a complementary feedstock.

9. (canceled)

10. (canceled)

11. (canceled)

12. The process of claim 6, further comprising the step of fabricating a tire component with the elastomeric polymer.

13. A method for producing an elastomeric polymer, the method comprising:
(a) providing styrene monomer synthesized from constituents of a gaseous stream that is obtained from the gasification of a feedstock including carbonaceous materials; and
(b) polymerizing the styrene with diene monomer to form an elastomeric polymer.

14. The method of claim 13, where the gaseous stream includes carbon monoxide, hydrogen, and carbon dioxide, and wherein the styrene monomer is formed by converting at least a portion of the carbon monoxide, hydrogen, and carbon dioxide to ethanol; converting at least a portion of the ethanol to ethylene; reacting at least a portion of the ethylene to benzene to form ethylbenzene; and converting at least a portion of the ethylbenzene to styrene.

15. The method of claim 13, where greater than 50 mol %, or 60 mol %, or 70 mol %, or 80 mol %, or 90 mol %, or 99 mol % of the styrene mer units of the elastomeric polymer are obtained by polymerizing the styrene monomer or from the styrene and sustainable comonomer.

16. (canceled)

17. The method of claim 13, where the carbonaceous material includes used tire feedstock, biomass, solid municipal waste, or a mixture of tire feedstock and a complimentary feedstock.

18. (canceled)

19. (canceled)

20. (canceled)

21. A tire component comprising:
a cured rubber matrix with filler dispersed therein, where the cured rubber matrix includes greater than 40 wt %, or 50 wt %, or 60 wt %, or 70 wt %, or 80 wt %, or 90 wt %, or 99 wt % of a crosslinked circular synthetic rubber that is obtained by polymerizing monomer synthesized from constituents of a gaseous stream that is obtained from the gasification of a feedstock including carbonaceous materials.

22. The tire component of claim 21, where the circular synthetic rubber is polybutadiene, and where the monomer that is synthesized from constituents of a gaseous stream is 1,3-butadiene.

23. The tire component of claim 21, where the circular synthetic rubber is a polybutadiene copolymer, and where the polybutadiene copolymer is obtained by the copolymerization of the 1,3-butadiene monomer and a sustainable comonomer.

24. The tire component of claim 21, where the carbonaceous material includes used tire feedstock, biomass, solid municipal waste, or a mixture of used tire feedstock together with a complementary feedstock.

25. (canceled)

26. (canceled)

27. (canceled)

28. The tire component of claim 21, where said tire component includes one or more additional constituents dispersed within the cured rubber matrix and one or more of these additional constituents are sustainable materials, and where greater than 40 wt %, or 50 wt %, or 60 wt %, or 70 wt %, or 80 wt %, or 90 wt %, or 99 wt % of the tire component includes circular synthetic rubber and sustainable materials.

29. The tire component of claim 21, where the filler includes recycled carbon black, rice husk ash derived silica and pyrolysis carbon black, or a sustainable oil dispersed within said rubber matrix.

30. (canceled)

31. (canceled)

32. A tire including the tire component of claim 21, where the tire includes greater than 40 wt %, or 50 wt %, or 60 wt %, or 70 wt %, or 80 wt %, or 90 wt %, or 99 wt % sustainable material, or recycled metal.

33. (canceled)
